(19) United States
(12) Patent Application Publication
(10) Pub. No.: US 2025/0204981 A1
(43) Pub. Date: Jun. 26, 2025

(54) DEVICE AND METHOD FOR PREVENTING AND TREATING A VASOSPASM

(71) Applicant: phenox GmbH, Bochum (DE)

(72) Inventors: Hans HENKES, Stuttgart (DE); Ralf HANNES, Dortmund (DE); Hermann MONSTADT, Bochum (DE)

(73) Assignee: phenox GmbH, Bochum (DE)

(21) Appl. No.: 19/077,464

(22) Filed: Mar. 12, 2025

Related U.S. Application Data
(63) Continuation of application No. 16/329,869, filed on Mar. 1, 2019.

(30) Foreign Application Priority Data
Sep. 8, 2016 (DE) DE102016116871.8

(51) Int. Cl. A61B 18/14; A61B 18/00; A61N 1/05; A61N 7/00; A61N 7/02

(52) U.S. Cl. CPC A61B 18/1492; A61N 1/0529; A61N 7/022; A61B 2018/0016; A61B 2018/00214; A61B 2018/00404; A61B 2018/00446; A61B 2018/00875; A61N 2007/003

(57) ABSTRACT

A device having a stent structure with electrical conductors via which electrical pulses, high-frequency pulses or ultrasonic pulses can be applied to nerve fibers extending in the vascular wall of the blood vessel in order to temporarily or permanently reduce the function of the nerve fibers so as to allow prevention or treatment of vasospasm.

This application is a continuation of U.S. application Ser. No. 16/329,869, filed Mar. 1, 2019, pending, which is a national stage of PCT application PCT/EP2017/072451, filed Sep. 7, 2017, which claims priority to German application DE102016116871.8, filed Sep. 8, 2016.

### FIELD OF THE INVENTIONS

The invention relates to an apparatus or device having a stent structure intended for the insertion into intracranial blood vessels of the human or animal body, wherein the stent structure having an expanded state in which it is capable of getting into contact with the inner wall of the blood vessel and a compressed state in which the stent structure being located in a microcatheter can be moved through the blood vessel, wherein the stent structure is connected to an insertion aid and wherein the stent structure is capable of automatically transitioning to the expanded state upon release from the microcatheter. The device is used to prevent or treat vasospasm. Moreover, the invention also relates to a relevant method.

A spasmodic constriction of a blood vessel is known as vasospasm. Vasospasms involve the risk of blood no longer being supplied in sufficient quantities to downstream vessels (ischemia) which may lead to necrosis of the tissue thus cut off from perfusion. Especially in the cerebral area, vasospasm can occur a few days after subarachnoid hemorrhage (SAH), quite frequently as a result of the rupture of an aneurysm. Other causes of subarachnoid hemorrhage are craniocerebral traumata and bleeding from vascular malformations or tumors. Blood that has ingressed into the subarachnoid space washes around the vessels located there and is regarded as the most important triggering factor of vasospasm. About 60% of all SAH patients experience a more or less pronounced vasospasm occurring roughly between the fifth and twentieth day after bleeding. If the arterial vessels are severely constricted, the dependent brain tissue becomes undersupplied and may suffer irreversible damage (cerebral infarction). Approx. 15 to 20% of all patients who primarily survived SAH experienced permanent neurological damage with resulting disability. Approximately 5% of the initially surviving SAH patients subsequently die as a result of cerebral Vasospasm. In this respect, vasospasm is one of the main reasons for apoplexy in this region or even mortalities occurring after rupturing of an aneurysm and/or bleeding from it or as a result of an operation.

Usually, vasospasm is treated with medication, in particular calcium channel blockers or drugs are put to use that cause the NO level in the blood to increase. An example of a calcium channel blocker is nimodipine which is frequently used after subarachnoid bleeding with a view to preventing vasospasms. However, such a medication-based treatment is associated with significant side effects and, moreover, is both cost-intensive and time-consuming.

Further possibilities for the treatment of vasospasm are intensive medical measures such as raising the arterial blood pressure and increasing the circulating blood volume, widening narrowed vessels with the help of a balloon, blocking the stellate ganglion, and the surgical elimination of sympathetic nerve fibers (sympathicolysis). These treatment methods are individually inconsistent in their effectiveness, sometimes very complex, and often not effective for a sufficiently long period of time. Surely, the blockade of the stellate ganglion as well as the operative sympathicolysis are to be considered effective because of the sympathetic nerve fibers in the wall of the cerebral arteries being essentially involved in the development of cerebral vasospasm. However, these methods are nevertheless insufficient to completely prevent and treat cerebral vasospasm, as the blockade of the stellate ganglion lasts only a few hours and an operative sympathectomy is limited to a narrowly defined vascular segment only, which must even be surgically prepared for this purpose.

It is thus the objective of the present invention to provide means that allow prophylaxis and treatment of vasospasm in some other way.

As proposed by the invention, this objective is achieved by an apparatus/device having a stent structure which is intended for insertion into intra-cranial blood vessels of the human or animal body, wherein the stent structure has an expanded state in which it is capable of abutting the inner wall of the blood vessel and a compressed state in which it is movable through the blood vessel when the stent structure is inside a microcatheter, wherein the stent structure is connected to an insertion aid and wherein the stent structure is capable of automatically transitioning into the expanded state upon release from the microcatheter, wherein the stent structure has electrical conductors via which electrical pulses, high-frequency pulses or ultrasonic pulses can be applied to nerve fibers extending in the vessel wall of the blood vessel in order to temporarily or permanently reduce the function of the nerve fibers so as to allow prevention or treatment of vasospasm.

The invention is therefore based on the use of a stent structure for the endovascular denervation of brain-supplying arteries. Endovascular procedures for the denervation of sympathetic nerve fibers are known in the field of denervation of the renal artery, but this serves to interrupt nerve fibers between the brain and kidney in order to reduce the release of substances that increase blood pressure. What is more, balloon catheters used for this purpose are not suitable for use in the intracranial area.

Physically, pulses can be applied to the nerve fibers in the form of high-frequency (HF) signals, direct current, alternating current or ultrasound. As a rule, denervation is ultimately based on heating of the vessel wall, which leads to the elimination or impairment of the function of the nerve fibers. The use of high-frequency or ultrasound pulses is preferred in so far as this allows energy maxima to be generated in the depth of the surrounding vessel wall, so that specifically the nerve fibers are damaged and not the entire vessel wall. The nerve fibers involved here are those of the sympathetic nervous system.

A single impulse application to the nerve fibers typically lasts for a period of 30 to 120 seconds, whereby the nerve fibers can be heated to a temperature ranging between 5° and 80° C. The penetration depth of the energy into the vessel wall, for example, ranges between 1 and 3 mm. In the event of HF pulses, the frequency typically is 300 to 4000 kHz. High frequency within the meaning of this invention denotes electromagnetic waves having a frequency>1 kHz, including microwaves with a frequency higher than 1 GHz.

Stents, also known as vascular endoprostheses, are often employed for the treatment of vasoconstrictions and are permanently implanted at the location of the constriction with a view to keeping the vessel lumen open. Typically, stents have a tubular structure and are either produced by laser cutting to achieve a surface consisting of struts with openings between them or they consist of a wire braiding. Stents can be moved by means of a catheter to the placement site where they are expanded; in the event of self-expanding stents made of shape-memory material, expansion and contact with the inner wall of the vessel take place automatically. After final placement, only the stent remains at the target site; catheter, guide or pusher wires, and other auxiliary means are removed from the blood vessel system. Implants of similar design having a higher surface density, so-called flow diverters, are also used for the occlusion of aneurysms in that they are placed in front of the neck of an aneurysm. However, the prevention or treatment of vasospasm with the help of a stent structure has not yet been described.

### SUMMARY

The inventive device serves the endovascular denervation of brain-supplying arteries for the prevention and treatment of vasospasm caused by bleeding. The device is particularly flexible and for that reason can be inserted into arteries inside the skull. The device changes the arterial flow of blood to such a minor extent only that there is no risk of an undersupply of the brain. The device can be used only once, remain in the vessel to be treated for several days or be implanted on a permanent basis.

The effect of the device proposed by the invention is based on a functional reduction or interruption of nerve fibers in the vascular wall of the affected blood vessels. This can range from a temporary reduction in function to a permanent destruction/elimination of the nerve fibers. In order to reduce/interrupt the function of the nerve fibers, energy is transferred from the device to the vessel wall, whereby transmission of energy takes place by means of electrical pulses, high-frequency pulses or ultrasonic pulses. These result in at least partial atrophy/sclerosis of the nerve fibers. Energy is transferred to the vessel wall by means of electrodes or ultrasonic transmitters, whereby the supply of energy to the electrodes/ultrasonic transmitters is brought about by means of electrical conductors which are part of the stent structure. The electrodes, which usually constitute the ends of the electrical conductors, are normally enlarged compared to the conductor proper. For example, the electrical conductors may be provided with round or square enlarged end sections that act as electrodes.

The stent structure is of self-expanding type, that is, after liberation from a microcatheter, in which it is advanced to the target site, it independently assumes without external influence an expanded state causing it to attach itself to the inner wall of the affected blood vessel. Moreover, the transition from the compressed to the expanded state should be reversible, i.e. the stent structure should be able to be transferred from the expanded state back to the compressed state, in particular to enable it to be withdrawn into the microcatheter after use and in this way removed from the blood vessel system. Self-expanding stent structures of this type are basically well known in the state of the art, for example with a view to keeping blood vessels open permanently in the event of vascular constrictions caused by arteriosclerosis. An advantage of a self-expanding stent structure is that it can be particularly filigree due to the fact that additional means such as balloons required for expansion can be omitted. Typically, self-expanding stent structures are made of a material having shape memory properties, especially shape memory metals such as nickel-titanium alloys. In this context, nitinol is used quite frequently. However, also conceivable are polymers having shape-memory characteristics or other alloys.

The insertion aid is typically a pusher wire, also known as guidewire. Such pusher wires are also used in a similar manner for the placement of implants that are intended to remain permanently in the vessel system in which case, however, the pusher wire is connected to the implant via a severance point, and said severance point may be designed for a mechanical, thermal, electrolytic or chemical detachment. On the other hand, the device pursuant to the invention is usually only temporarily navigated to the target position in order to apply energy to the vessel wall. The insertion aid is preferably made of stainless steel, nitinol or a cobalt-chromium alloy. However, also conceivable is a device that has a stent structure designed for permanent placement in the vascular system, i.e. said structure having a detachment point located between the insertion aid and the stent structure. As a rule, the stent structure is connected to the insertion aid at its proximal end, although other connections between the insertion aid and the stent structure are not precluded.

The insertion aid or pusher wire is preferably attached radially outward to the proximal end of the stent structure. In other words, the connection between insertion aid and stent structure is not in the center of the stent structure but arranged eccentrically at or near the inner wall of the vessel. In this manner, the flow of blood is impeded to a minor degree only. What is more, an eccentric arrangement of the insertion aid facilitates retraction of the device into the microcatheter.

Usually, treatment is carried out in such a way that the inventive device arranged inside a microcatheter is moved towards the placement site, i.e. the location where vasospasm has occurred or the place where vasospasm is likely to occur. Following this, the microcatheter is retracted in proximal direction causing the deployment of the stent structure which now expands and touches the inner wall of the vessel. Pulses are then applied to the nerve fibers in the vascular wall which may take place repeatedly, even over longer periods of several hours or several days. Finally, the microcatheter is again moved in distal direction with a view to embracing the stent structure following which the microcatheter together with the device is retracted. The treatment described here may be repeated on several days in succession.

The terms “proximal” and “distal” are to be understood such that they refer as proximal to parts that point towards the attending physician when inserting the device, and as distal to parts that point away from the attending physician. Typically, the device is thus moved forward in distal direction with the aid of a microcatheter. The term “axial” refers to the longitudinal axis of the device extending from proximal to distal while the term “radial” denotes levels/planes extending vertically thereto.

A treatment undertaken with the device proposed by the invention may at the same time be accompanied by a medication-based treatment, for example using nimodipine. This can be applied intraarterially at the site where a treatment or prevention of vasospasm is envisaged.

Basically, the stent structure may consist of individual, interconnected struts. Such a stent structure can be manufactured in a known manner by laser cutting technique. Moreover, it is thought expedient to process the stent structure by electropolishing to make it smoother and rounder and thus render it less traumatic. This also reduces the risk that germs or other impurities may adhere to the structure.

Alternatively, the stent structure may also be a mesh-like structure consisting of individual wires in the form of a braiding. The wires in this case typically extend helically along the longitudinal axis, with intersecting opposed wires extending above and below each other at points of intersection resulting in honeycomb-like openings being created between the wires. The total number of wires preferably ranges between 8 and 64. As wires forming the mesh structure individual wires made of metal may be employed but it is also possible to provide strands, i.e. several wires of small diameter arranged so as to form a filament, preferably twisted around each other.

An advantage of a stent structure comprising interconnected struts that in particular are produced by laser cutting techniques over a mesh structure consisting of wires is that during the expansion process a strut comprising stent structure will be less prone to longitudinal contraction than a mesh structure. Longitudinal contraction should be kept to a minimum because the stent structure exerts additional stress on the surrounding vessel wall during longitudinal contraction. Due to the fact that vasospasm is especially caused by stimuli acting on the vessel any additional stress has to be avoided in the treatment of vasospasm.

The struts or wires may have a round, oval, square or rectangular cross section, with the edges being advantageously rounded off in the event of a square or rectangular cross section. When braces or wires of an essentially rectangular cross section are put to use, it has turned out to be of advantage to provide struts/wires of a height and width of between 20 and 300 μm, preferably between 20 and 70 μm, with a rectangular cross section the edges of which are rounded also being considered as essentially rectangular. In the event of a round cross section the diameter should range between 20 and 300 μm. Irrespective of whether struts or braided wires are used, it is important that electrical conductors are provided in order to be able to carry out the application of pulses to the nerve fibers. The electrical conductors may be the struts/wires themselves, the conductors may be connected to the struts/wires, or the conductors may be separate components of the stent structure.

According to a preferred embodiment, the stent structure is provided with a spine extending from proximal to distal from which struts originate and form the circumference of the stent structure in the expanded state. For example, the stent structure may resemble a human spine, with the struts originating from the spine are comparable to the ribs. In particular, the struts originating from the spine can substantially form a ring when they are in the expanded state so that they are in contact over the entire or large portions of the circumference with the substantially circular inner wall of the blood vessel when viewed in cross-section. Notably, two struts can each form open rings having a gap. The connection points between the struts and the spine can also be offset from each other; this reduces the risk of electrical conductors of the struts touching each other and producing a short circuit.

The struts originating from the spine and forming open rings may also be composed of two or more partial struts, i.e. two or more partial struts starting from the spine run parallel to each other and terminate in a common end point. Between the end points, which are formed by oppositely arranged groups of partial struts, a gap is thus formed. In the event a strut is made up of two partial struts, the embodiment can also be described in such a way that the two partial struts together form an arc on the spine, with the vertex of the arc corresponding to the aforementioned end point.

Irrespective of the embodiment, in which the struts are originating from a common spine, struts can also be made up of parallelly extending partial struts. A stent structure comprising several narrower partial struts can unfold more reliably radially than a stent structure with wider struts when the stent structure is freed from the external constraint of the microcatheter.

The angle that the struts originating from the spine form with respect to the spine can be a right angle, but deviations from the right angle may also be provided, for example an extension to some extent in the proximal or distal direction. The angle formed at the connection point between the struts and the spine can therefore range between 30° and 90° in the expanded state, with the struts pointing both in distal and proximal direction. More typical, however, are embodiments in which the struts point in the distal direction. The struts can be provided with electrical conductors to allow a pulse to be transmitted to the nerve fibers. It may be sufficient if only individual struts originating from the spine have electrical conductors; however, it is also possible to equip all struts with electrical conductors. Furthermore, the number of struts originating from the spine can be optionally selected, but the minimum number of struts is one.

The electrical conductors should converge at the proximal end of the stent structure and be connected to the insertion aid. Over the length of the insertion aid, there is usually an electrical connection between the electrical conductors and a current source that is typically located outside the body. This ensures that electrical or other impulses can be transmitted under external control through the stent structure. However, a power source that is part of the device itself would also be conceivable in principle but in this case such a power source would have to be particularly compact to be capable of being inserted into intracranial blood vessels. The number of electrical conductors may vary depending on whether a single pair of electrodes/an ultrasonic transmitter or multiple pairs of electrodes or ultrasonic transmitters are employed. On the one hand, the provision of several electrical conductors is to be seen advantageous in that it allows impulses to be applied at various places on the inner wall of the vessel, possibly simultaneously. On the other hand, however, care must be taken to ensure that the entire device/apparatus remains sufficiently flexible to be able to be navigated through intracranial blood vessels of narrow lumen.

The individual electrical conductors should be electrically insulated from each other in order to avoid short circuits. This is especially true if the struts or wires forming the stent structure are arranged relatively close to each other. It may be sufficient for the electrical conductors to be electrically isolated only in those areas where they are closely spaced to one another, for example at the proximal end of the stent structure where the electrical conductors transition into the insertion aid, but in principle it is advantageous for the electrical conductors to be fully isolated, except where appropriate for the areas of the electrical conductors through which the pulse application is made, i.e. as a rule at the ends where the electrodes are arranged.

It is also possible to make use of an embodiment in which the electrical conductors are electrically insulated, but where no electrical insulation is deliberately provided in some places with a view to enable pulses to transmitted at these points or between these points. Such an embodiment is particularly suitable for a stent structure comprising a mesh structure of individual wires forming a braiding. In this case, the expansion of the stent structure ensures quasi automatically that the wires, at least some of which can simultaneously perform the function of electrical conductors, are in contact with the inner wall of the vessel, which also applies to areas/places of the electrical conductors where insulation has been dispensed with. One advantage of such a stent structure is that it can be used for different blood vessels having different diameters.

Moreover, preference is given to a device in which pairs of electrical or high frequency (HF) electrodes are arranged on the periphery of the stent structure in such a manner that the electrodes in the expanded state and implanted in the blood vessel are spaced apart by a gap so that an applied current flow to the electrodes across the gap acts on the inner wall of the blood vessel. The pulse can be an electric or a high frequency pulse. In particular, the embodiment can be combined with the embodiment described hereinbefore, in which the stent structure is provided with a spine extending from proximal to distal, from which struts provided with electrical conductors originate. In this way, pairs of struts can originate from the spine with the electrodes being arranged at the ends of the relevant strut pairs. In view of the small size of the stent structure as a whole, the spacing between the electrodes is of course also small and usually amounts to ≤1 mm.

It is also considered useful if the electrodes are provided with a radiopaque marking. In this way, the attending physician can see whether the electrodes are still a short distance apart, as desired, or whether they touch each other, i.e. causing a short circuit to occur. Since the struts even in the expanded state are subject to a radial force exerted by the inner wall of the vessel, the extent to which the stent structure is compressed also depends on the inner diameter of the blood vessel. For example, a certain stent structure can be used with a sufficiently large blood vessel because a sufficiently large gap exists between the electrodes, whereas with blood vessels of smaller size there may be contact between the electrodes due to the greater compression the stent structure is exposed to, resulting in the transmission of pulses being ruled out. Different stent structures should therefore be available for use with blood vessels having different inner diameters.

Another conceivable embodiment provides for a bridge made of insulating material to be arranged between the electrodes. In this way, a short circuit is effectively prevented. If the insulating material additionally has a certain flexibility, the stent structure will be capable of adapting to the inner diameter of a blood vessel so that a given stent structure can be used in blood vessels of different size.

It is considered particularly advantageous if the electrodes have a radiopaque core inside which can serve as a marking. One or more radiopaque markers may also be arranged at other positions on the device to allow the attending physician to visualize the placement and deployment of the device. The radiopaque markers may, for example, consist of platinum, palladium, platinum-iridium, tantalum, gold, tungsten or other metals opaque to radiation. Appropriate radiopaque markers at the ends of the stent structure, especially at the distal end, are particularly useful. It is also possible to provide the struts or wires of the stent structure with a coating consisting of a radiopaque material, such as a gold coating. This coating can, for example, have a thickness of 1 to 6 μm. Such a gold coating can be used additionally or instead of the radiopaque markers.

It is thought expedient to provide the stent structure with several pairs of electrodes that can generate an electrical or high-frequency pulse. In this way, an impulse can be applied at several locations of the vessel wall, with the application or transmission taking place simultaneously or consecutively. This is important because the vascular wall often contains several nerve fibers whose denervation is important for the success of the treatment.

When viewed in the longitudinal direction of the stent structure, the electrodes, pairs of electrodes and/or ultrasonic transmitters (generally: pulse generators) can be arranged circumferentially offset from each other. In other words, the electrodes, which are arranged from proximal to distal in different segments of the stent structure, also act on different segments with regard to the inner circumference of the blood vessel. In cross sectional view, a pulse generator may, for example, be located at 12:00 o'clock, a pulse generator at 3:00 o'clock, a pulse generator at 6:00 o'clock and another pulse generator at 9:00 o'clock position. Such an embodiment offers the advantage that, without having to rotate the stent structure, different nerve fibers can be processed that extend in the longitudinal direction in the vessel wall. If necessary, the stent structure can be advanced distally or retracted proximally in order to bring specific pairs of electrodes to different circumferential positions of the vessel wall and allow the pulses to take effect there. This is important because advancing or retracting the device and thus the stent structure is comparatively easy, but a rotation of the stent structure is difficult to achieve since the device as a rule has been advanced over considerable distances into the intracranial region, which makes the exertion of torsional forces considerably more difficult.

Of advantage is an embodiment in which the stent structure comprises a plurality of substantially annular elements which are spaced in the longitudinal direction of the device, each element comprising two electrical conductors that belong to an electric circuit, the two electrical conductors each terminating in one electrode and the two electrodes being separated from each other by a gap when the device is implanted in the blood vessel in the expanded state. In particular, said embodiment can be combined with the embodiment described hereinbefore, in which struts originate from a spine extending from proximal into distal direction, wherein in the present case a strut originating from the spine extends in a first direction with a first electrical conductor and a second strut originating from the spine extends in a second direction with a second electrical conductor. The stent structure thus resembles a human spine with ribs, with the ends being separated by a gap. This structure has therefore not a closed, but an open ring shape. As described above, the gaps between the individual struts may be arranged offset from each other for different strut pairs. It is also possible to fill the gaps with an electrically insulating material.

It is appropriate for the device, preferably the stent structure, to be provided with means for measuring electrical resistances, in particular means for measuring impedance, i.e. the measurement of alternating current resistance. Such a resistance measurement is important insofar as different tissues may have different electrical resistance. To be able to determine the amount of energy to be applied for the denervation of certain nerve fibers, a resistance measurement is therefore considered useful. On the basis of the resistance value so detected, a data matrix can be built, for example, to determine which defined current-voltage signal can appropriately be used to achieve the desired effect, for example, to induce a specific temperature. After the treatment, the success of the treatment can be checked by performing another resistance measurement.

The resistance measurement does not necessarily have to be integrated into the device proposed by the invention, i.e. a separate device for resistance measurement is also conceivable.

The stent structure can be permeable, that is, have openings in the radial direction, but it is also possible to provide a stent structure with a membrane on the inside, i.e. the luminal side. On the abluminal side, however, the surface of the device comes into direct contact with the inner wall of the vessel. In this case the membrane serves to separate the vessel lumen from the usually metallic wires or struts of the stent structure. The membrane can also produce some electrical insulation of the electrical conductors in the luminal direction. On the other hand, when the stent structure is present in a compressed state the provision of an additional membrane requires additional space so that such a stent structure is less compactly foldable.

Normally, the stent structure is of open design at the proximal end. At its distal end, the stent structure may also be open but can also be of closed design. A stent structure that is open at both ends offers the advantage that the blood flow is impeded as little as possible so that an undersupply of downstream blood vessels and tissue they supply with blood can be prevented. On the other hand, providing the distal end with a closed structure is more atraumatic. It is to be noted that referring to an open structure means no struts or wires are arranged at the respective end of the stent structure and that struts/wires are only arranged over the outer circumference of the stent structure. In the event of a closed end, however, struts or wires also exist in the center of the stent structure. However, since there are still openings between the struts or wires, even a closed distal end is not completely impervious and still allows the flow of blood through the respective openings.

An antithrombogenic coating of the stent structure is considered expedient. Such a coating an be applied to the entire stent structure or only to the inside of it because the structure remains within the blood vessel for a certain time span during which the prevention of clots is mandatory that might form in the vessel already constricted due to vasospasm that has occurred. The outside of the stent structure could advantageously be coated with an agent conducive to vessel relaxation, for example with a calcium channel blocker such as nimodipine.

Typically, the diameter of the stent structure in freely expanded state is in the range of between 2 and 8 mm, preferably ranges between 4 and 6 mm. The total length of the stent structure in expanded state as a rule amounts to 5 to 50 mm, preferably lies between 10 and 45 mm, further preferred between 20 and 40 mm In the case of a stent structure consisting of struts the structure can be cut, for example, from a tube having a wall thickness of between 25 and 70 μm; in the case of a mesh structure consisting of interwoven wires, the preferred wire thickness is 20 to 70 μm. For example, a microcatheter by means of which the device can be navigated to its target site in a compressed state has an internal diameter of between 0.4 and 0.9 mm.

In addition to the device the invention proposes, the invention also relates to a method for the prevention or treatment of vasospasm. Said method provides for the stent structure of the inventive device to be transported to the target site in the blood vessel by means of the insertion aid and expanded there, which as a rule is achieved by retracting the microcatheter accommodating the device, said retraction taking place in proximal direction. Subsequently, electrical pulses, high-frequency pulses or ultrasound pulses are applied to nerve fibers extending in the vascular wall of the blood vessel. If thought appropriate, the application of pulses may be repeated several times. During this process of applying the individual pulses, the stent structure can remain at a given position, be advanced distally or retracted proximally to act on different nerve fibers as required. As a rule, the stent structure is moved when situated inside the microcatheter, since the risk of injury to blood vessels would otherwise be too high, especially when the expanded stent structure is advanced. In any case, an injury or excessive irritation should be avoided, as this may be a causal factor for the occurrence of vasospasm. Therefore, advancing or retracting the stent structure to reach another longitudinal position is brought about in such a way that initially the microcatheter is advanced in order to transfer the stent structure into its compressed state with the stent structure thus being accommodated in the microcatheter, followed by the microcatheter and thus also the stent structure located within the microcatheter being navigated to the desired position where the stent structure is finally released again from the microcatheter.

The entire device may also be removed temporarily from the blood vessel system and reinserted later with a view to continuing the treatment over several days, for example. For reasons of sterility, however, a new device must normally be used for each treatment. As a rule, the device is removed from the blood vessel system by pushing the microcatheter distally over the released stent structure, whereupon it folds up again and can be retracted in proximal direction together with the microcatheter.

Any and all statements made with respect to the device shall equally apply in the same way as well to the method and vice versa.

### BRIEF DESCRIPTION OF THE DRAWINGS

Further elucidation of the invention is provided by way of example through the enclosed figures where

FIG. 1 is a side view of a device proposed by the invention;

FIG. 2 illustrates the stent structure of the inventive device shown in FIG. 1;

FIG. 3 is a partial view of the illustration shown in FIG. 2;

FIG. 4 shows an alternative stent structure;

FIG. 5 shows part of a stent structure having several electrical conductors;

FIG. 6 shows electrodes of the stent structure and

FIG. 7 illustrates a stent structure in developed form.

### DETAILED DESCRIPTION

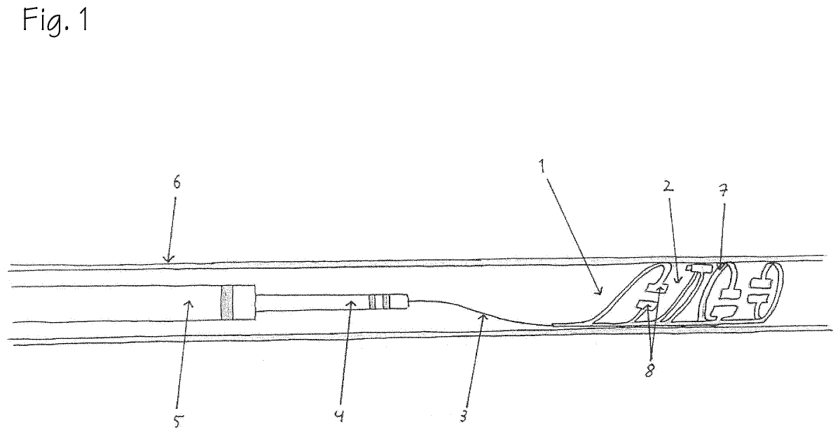

In FIG. 1 a device 1 according to the invention is shown in side view which is situated inside a blood vessel 6. Device 1 has a stent structure 2 and is provided with an insertion aid 3 in the form of a guidewire. The stent structure 2 is shown here in its expanded form implanted into the blood vessel 6. The stent structure 2 is advanced within the microcatheter 4 from proximal (here: left) to distal (here: right); by advancing the microcatheter 4 or withdrawing the stent structure 2, the structure folds up again so tightly that it can be accommodated in the microcatheter 4 for removal out of the blood vessel system together with the microcatheter. The microcatheter 4 itself is guided through another catheter 5 having a larger lumen.

The stent structure 2 is provided with struts 7, which are essentially ring-shaped in pairs and are intended to be placed in position against the inner wall of the blood vessel 6. In addition, the struts 7 are provided with electrical conductors which are electrically connected to the electrodes 8 located at the end of the struts 7. For each ring of struts 7 there are pairs of electrodes 8 that belong to each other, with a small gap being provided between them via which an impulse, for example an electrical or HF pulse, can be applied to the surrounding tissue. Furthermore, it can be seen that for the individual rings formed by the struts 7 the electrode pairs 8 and thus also the gap between the electrodes are offset from one another with respect to their position in the circumference of the blood vessel, that is, different rings of struts 7 apply pulses at different radial positions.

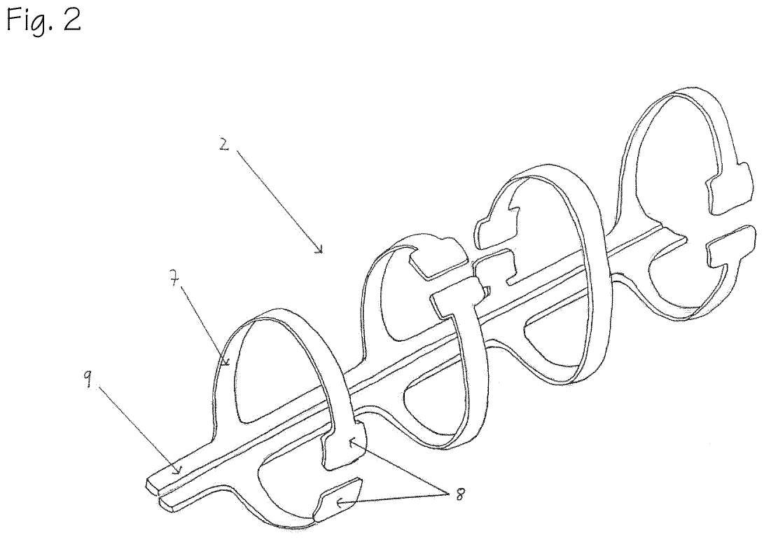

FIG. 2 is an enlarged view of the stent structure 2 depicted in FIG. 1. It can be seen that the struts 7 each in pairs form an open ring, with an electrode 8 being arranged at the end of each strut 7. For the individual rings of struts 7, which are arranged one behind the other in the longitudinal direction, the electrodes 8 are arranged offset from each other. The pulses, which are intended to act on different radial areas of the wall of the blood vessel and the nerve fibers running therein, can be emitted simultaneously, but said emission may also take place offset in time. For a certain ring of struts 7, the position where the pulse application is to take place may, for example, also be selected by appropriately displacing the stent structure 2 in longitudinal direction.

Struts 7 originate from a common spine 9 that runs in the longitudinal direction of stent structure 2. In the configuration shown here, the spine 9 itself can be divided in two so that one half of the spine 9 serves to supply power to the first half of the struts 7 while the second half of the spine 9 serves for supplying power to the second half of the struts 7.

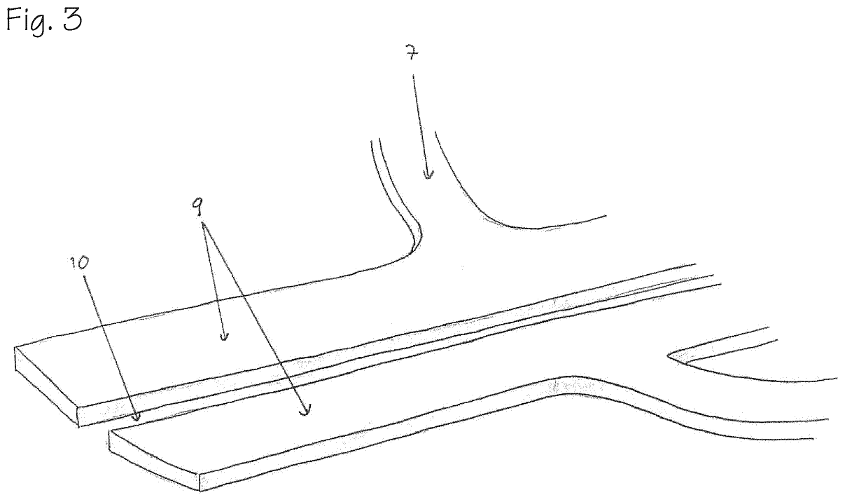

A partial section of the stent structure 2 depicted in FIG. 2 is shown in FIG. 3; it can be seen how the struts 7 are connected to the spine 9 and that there is an insulation 10 between the two halves of the spine 9 which ensures that no short circuit occurs between the two halves of the spine 9.

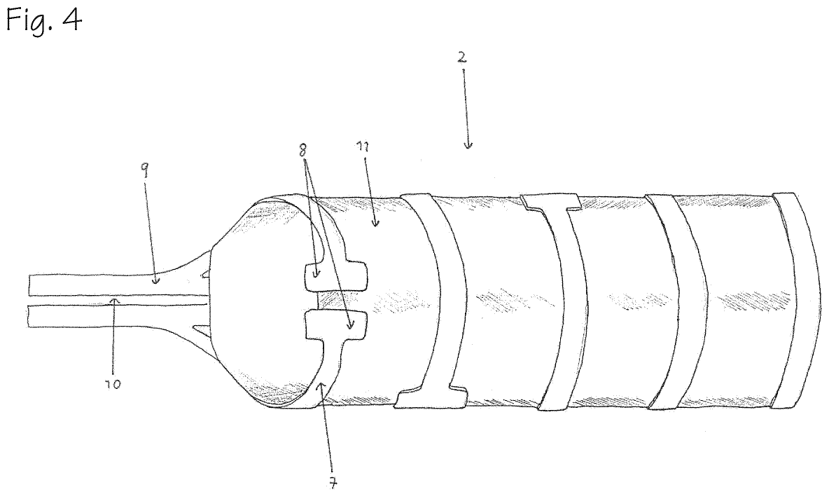

FIG. 4 shows a stent structure 2 which is basically similar to the stent structure 2 illustrated in FIG. 2, but in which a membrane 11 is arranged on the luminal side, i.e. inside the stent structure 2, said membrane separating the actual lumen of the blood vessel 6 from the stent structure 2 and thus creates an isolation in the luminal direction.

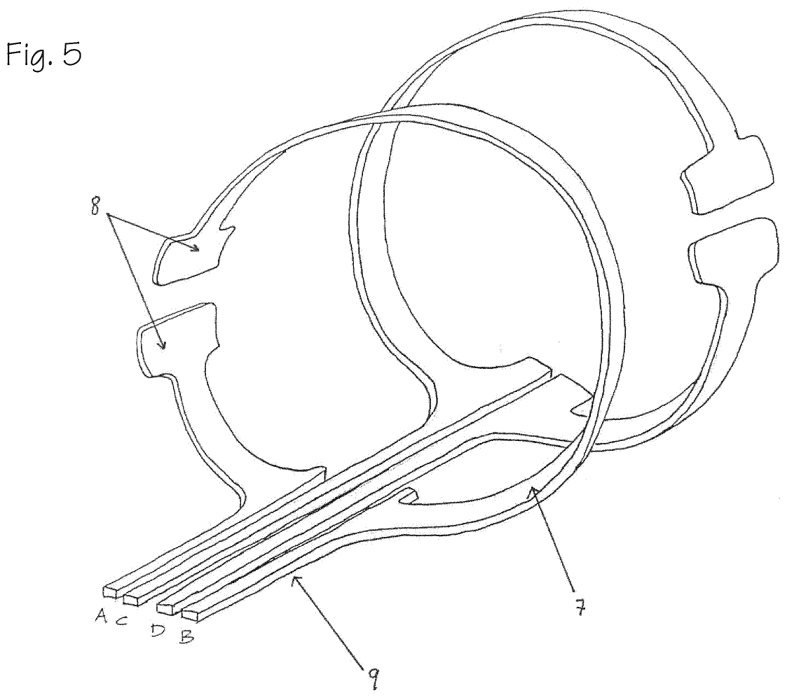

In FIG. 5 two open rings are illustrated that are arranged one behind the other in the longitudinal direction and formed by struts 7, each originating from the spine 9. The latter is provided with 4 conductors A, B, C, D which ensure a current supply to the electrodes 8. The power supply via conductors A, B on the one hand and C, D on the other hand may take place simultaneously or sequentially.

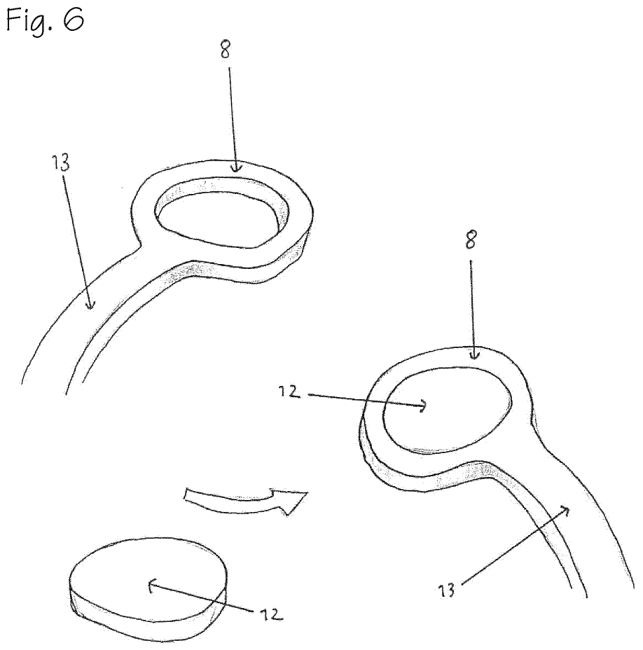

FIG. 6 shows electrodes 8 forming the ends of the electrical conductors 13. In the embodiment shown here, the electrical power is conducted directly via the struts 7 to the electrodes 8, i.e. the struts 7 are also electrical conductors 13. To enable electrodes 8 to be visualized, they have an opening on the inside into which radiopaque markers 12 are pressed. The radiopaque markers 12, for example, can be made of platinum or a platinum alloy. In the radiographic image, the attending physician thus immediately recognizes how the electrodes 8 are arranged, which emit the pulses essential for the treatment. Moreover, the physician can verify that no short circuit has occurred between electrodes 8.

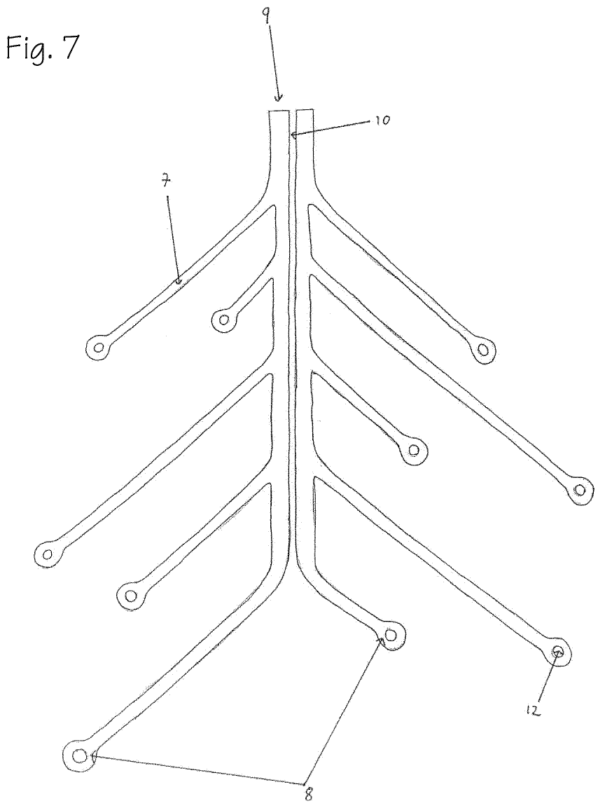

FIG. 7 depicts a stent structure 2 in developed form, that is, the struts 7 forming an open ring were pressed into a planar surface resulting in the two-dimensional representation shown. It can be seen that the struts 7 are of different lengths. In this manner it is achieved that after insertion into the blood vessel the electrodes 8 are finally arranged in an offset or staggered way on the inner wall of the blood vessel so that impulses are allowed to act on different sections/segments.

We claim:

1. A medical device for treating vasospasm of a blood vessel, the device comprising:
an elongated body with a plurality of substantially annular elements spaced in the longitudinal direction of the elongated body;
wherein each of the plurality of substantially annular elements originates from a same longitudinal location of the elongated body; and
wherein each of the plurality of substantially annular elements comprises a gap with a pair of electrical conductors at the opposite side of the gap, so that an applied current flow to the pair of electrical conductors acts across the gap on the inner wall of the blood vessel when the device is placed against the inner wall of the vessel.

2. The medical device of claim 1, wherein the electrical conductors deliver an electrical pulse to the nerve fibres in the blood vessel in order to temporarily or permanently reduce the function of the nerve fibres.

3. The medical device of claim 2, wherein the electrical pulses are delivered simultaneously by all the electrical conductors of the device.

4. The medical device of claim 2, wherein the electrical pulses delivered consecutively by each pair of the electrical conductors on the same substantially annular element.

5. The medical device of claim 1, wherein each substantially annular element is in the shape of an open ring with two partial struts originating from the same longitudinal location of the elongated body, and the gap between the free ends of the two partial struts.

6. The medical device of claim 5, wherein each of the two partial struts of the same substantially annual element has an electrical conductor.

7. The medical device of claim 1, wherein the gaps of the plurality of substantially annular elements offset from one another.

8. The medical device of claim 1, wherein the electrical conductors are electrically insulated from one another.

9. The medical device of claim 1, wherein each pair of the electrical conductors of the same substantially annular element belongs to a same electric circuit.

10. The medical device of claim 1, wherein each electrical conductor of the device terminates in one electrode.

11. The medical device of claim 1, wherein the medical device comprises a mean for measuring electrical resistance.

12. The medical device of claim 1, wherein the medical device comprises a compressed state to allow the medical device to be placed inside a microcatheter.

13. A medical device for treating vasospasm of a blood vessel, the device comprising:
an elongate spine comprising a first elongate member, a second elongate member, and an elongate insulating member disposed between the first elongate member, a second elongate member, said elongate spine characterized by a length, a first end a second end; and
a first pair of struts comprising a first strut and a second strut, extending laterally from the spine, with the first strut joined to the first elongate member of the spine at a first location along the length of the spine, and with the second strut joined to the second elongate member of the spine at the first location of the spine along the length of the second elongate member, with the first strut extending in an arc from the first elongate member in a first direction, and the second strut extending in an arc from the second elongate member in a second direction opposite the first direction;
a second pair of struts comprising a third strut and a fourth strut, extending laterally from the spine, with the third strut joined to the first elongate member of the spine at a second location along the length of the spine, and with the fourth strut joined to the second elongate member of the spine at the second location along the length of the spine, with the third strut extending in an arc from the first elongate member in the third direction, and the fourth strut extending in an arc from the second elongate member in the fourth direction; wherein
the first strut extends arcuately toward the second strut, and the second strut extends arcuately toward the first strut, such that an end of the first strut distal to the first elongate member is proximate an end of the second strut distal to the second elongate member; and
the third strut extends arcuately toward the fourth strut, and the fourth strut extends arcuately toward the third strut, such that an end of the third strut distal to the first elongate member is proximate an end of the fourth strut distal to the second elongate member.

14. The medical device of claim 13, wherein the end of the first strut and the end of the second strut are spaced apart by a gap and the end of the third strut and the end of the fourth strut are spaced apart by a gap.

15. The medical device of claim 1, further comprising a current source configured to supply current to the struts through the first and second elongate members of the spine.

16. The medical device of claim 15, wherein an electrode is disposed on the end of each strut, and the current source is operably connected to each of the electrodes.

17. The medical device of claim 13, wherein the first strut has a first length and the second strut has a second length, and the third strut has third length and the fourth strut has a fourth length, wherein the first length is greater than the second length, and the third length is less than the fourth length, and the distal end of the first strut and the distal end of the second strut are separated by a first gap, and the distal end of the third strut and the distal end of the fourth strut are separated by a second gap.

18. The medical device of claim 13, wherein the first strut has a first length and the second strut has a second length, and the third strut has third length and the fourth strut has a fourth length, wherein the first length is greater than the second length, and the third length is less than the fourth length, and the distal end of the first strut and the distal end of the second strut are separated by a first gap, and the distal end of the third strut and the distal end of the fourth strut are separated by a second gap, such that the first gap and second gap are not aligned along the length of the spine.

19. The medical device of claim 13, wherein the struts join the spine at an angle between 30° and 90°.

20. A method of treating vasospasm of a blood vessel, said method comprising the steps of:
providing a medical device according to claim 13;
delivering the medical device into a blood vessel, proximate nerve fibers in a vascular wall of the blood vessel; and
applying electrical pulses to the nerve fibers in the vascular wall of the blood vessel surrounding the medical device, said step of applying electrical pulses comprising passing the electrical pulses through a gap between the end of the first strut and the end of the second strut; and applying electrical pulses through a gap between the end of the third strut and the end of the fourth strut, to treat vasospasm of a blood vessel.
